(19) United States
(12) Patent Application Publication
(10) Pub. No.: US 2026/0272418 A1
(43) Pub. Date: Sep. 17, 2026

(54) SYSTEMS AND METHODS FOR CONTROL OF ULTRASOUND SYSTEMS USING SPATIAL TRACKING

(71) Applicant: GE Precision Healthcare LLC, Waukesha, WI (US)

(72) Inventors: Jacob Bjerring Olesen, Copenhagen (DK); Fredrik Gran, Limhamn (SE); Henrik Jensen, Charlottenlund (DK)

(73) Assignee: GE Precision Healthcare LLC, Waukesha, WI (US)

(21) Appl. No.: 19/081,869

(22) Filed: Mar. 17, 2025

(51) Int. Cl. A61B 8/00; A61B 8/08

(52) U.S. Cl. CPC A61B 8/463; A61B 8/0841; A61B 8/4245; A61B 8/467

(57) ABSTRACT

An ultrasound imaging system includes a processing circuit including a processor coupled to a memory device storing instructions thereon that, when executed, cause the processing circuit to perform operations including receiving location data regarding the location of an external instrument with respect to a tissue imaged by a transducer, receiving image data of the tissue from the transducer, generating and displaying, based on the image data and the location data, an image on a display device of the ultrasound imaging system, the image including the tissue and a representation of the external instrument, receiving a user input via a user input element disposed on a probe of the ultrasound imaging system, the probe including the transducer, and controlling at least one of a display characteristic of the displayed image or an operating characteristic of the transducer based on the user input.

### FIELD

Embodiments of the subject matter disclosed herein relate to ultrasound imaging, and more particularly, to tracking the locations of tissues being imaged and instruments interfacing with the tissues.

### BACKGROUND

During a medical imaging scan, a plurality of medical images of a patient are obtained by a healthcare professional to measure or detect various aspects of anatomical features present within the medical images. When ultrasound images are obtained during surgery, the surgeon may exit the sterile field to capture the images or rely on a technician not in the sterile field to capture the images.

### SUMMARY

An embodiment relates to an ultrasound imaging system. The ultrasound imaging system includes a transducer configured to transmit and receive an ultrasound signal, a matching layer configured to have an acoustic impedance between a tissue to be imaged and a material of the transducer, a damping block configured to absorb ultrasound energy, an external instrument, a spatial tracking system configured to enable tracking of the transducer and the external instrument, and a processing circuit including a processor coupled to a memory device storing instructions thereon that, when executed, cause the processing circuit to perform operations including: receiving location data regarding the location of the external instrument with respect to tissue imaged by the transducer, receiving image data of the tissue from the transducer, generating and displaying, based on the image data and the location data, an image on a display device of the ultrasound imaging system, the image including the tissue and a representation based on the external instrument, receiving a user input via a user input element disposed on a probe of the ultrasound imaging system, the probe including the transducer, and controlling at least one of a display characteristic of the displayed image or an operating characteristic of the transducer based on the user input.

Another embodiment relates to a medical imaging system. The medical imaging system includes a processing circuit including a processor coupled to a memory device storing instructions thereon that, when executed, cause the processing circuit to perform operations including: receiving location data regarding the location of an external instrument with respect to a tissue imaged by a transducer, receiving image data of the tissue from the transducer, generating and displaying, based on the image data and the location data, an image on a display device of the medical imaging system, the image including the tissue and a representation based on the external instrument, receiving a user input via a user input element disposed on a probe of the medical imaging system, the probe comprising the transducer, and controlling at least one of a display characteristic of the displayed image or an operating characteristic of the transducer based on the user input.

Another embodiment relates to a method. The method includes receiving, by a processing circuit, location data regarding the location of the external instrument with respect to the tissue by the transducer, receiving, by the processing circuit, image data of the tissue from the transducer, generating and displaying, by the processing circuit, based on the image data and the location data, an image on a display device of the ultrasound imaging system, the image including the tissue and a representation based on the external instrument, receiving, by the processing circuit, a user input via a user input element disposed on a probe of the ultrasound imaging system, the probe including the transducer, and controlling, by the processing circuit, at least one of a display characteristic of the displayed image or an operating characteristic of the transducer based on the user input.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a diagram showing an ultrasound probe and external instrument, according to an example embodiment.

FIG. 3 is an illustration of an ultrasound image showing an intersection between an ultrasound probe and an external instrument, according to an example embodiment.

FIG. 4 is an illustration of an ultrasound probe including a user input element, according to an example embodiment.

FIG. 5 is an illustration of an ultrasound image showing a stored coordinate, according to an example embodiment.

FIG. 6 is an illustration of an ultrasound image showing a stored coordinate, according to another example embodiment.

FIG. 7 is an illustration of an ultrasound image showing a position of an ultrasound probe, according to an example embodiment.

FIG. 8 is a flow diagram illustrating a method of controlling an ultrasound system using spatial tracking, according to an example embodiment.

FIG. 9 is a flow diagram illustrating a method of controlling an ultrasound system using spatial tracking, according to another example embodiment.

FIG. 10 is a flow diagram illustrating a method of controlling an ultrasound system using spatial tracking, according to another example embodiment.

FIG. 11 is a flow diagram illustrating a method of controlling an ultrasound system using spatial tracking, according to another example embodiment.

### DETAILED DESCRIPTION

Referring generally to the figures, systems and methods for spatially tracking the location of an ultrasound probe and an external instrument are disclosed. More specifically, the systems and methods described herein include a spatial tracking system that tracks positions of the ultrasound probe and the external instrument. The probe includes a smart button that can be actuated to control a display of an ultrasound image (e.g., freezing the image, saving the image, etc.).

Ultrasound imaging systems may be used during surgery to assist with medical procedures. Specifically, the ultrasound imaging system (e.g., the processing circuit, display device, user interface, etc.) may be located within the operating room, but outside of a sterile field. Meanwhile, the surgeon or other medical professional that is imaging the patient by navigating the probe is located within the sterile field. Thus, to maintain sterility, the ultrasound system may need to be draped by a sterile cover to allow any interactions between surgeon and system. Even still, it may be challenging to have the ultrasound system placed within arm's reach of the operator, particularly in crowded operating rooms or when ultrasound system is placed on an opposite side of patient from where the surgeon is operating.

The surgeon may therefore have to exit the sterile field to adjust imaging settings, capture images, etc., or rely on another medical professional located outside of the sterile field to appropriately adjust the imagine parameters and control the system. This may reduce a workflow efficiency during surgery, increase imagining and surgery time, cause errors in the imaging procedure or to the medical procedure, etc.

Tracking of surgical instruments allows positioning of medical instruments in relation to each other while located inside of a patient's body and out of direction line of sight of medical personnel. A transducer of an ultrasound probe and an external instrument (e.g., a needle, trocar, etc.) are tracked. The tracked external instrument can be used as a guide to indicate if and where the instrument's calculated trajectory will intersect with the ultrasound image (e.g., based on the relative positions of the external instrument and the transducer). Various tracking technologies (e.g., spatial tracking systems) can be used to track the ultrasound probe and surgical instruments, such as electromagnetic (EM) tracking, optical tracking, acoustic tracking, radio frequency identification (RFID) tracking, etc.

Accordingly, the systems and methods described herein relate to ultrasound imaging using tracking that allows interaction between the operator located within the sterile field and the ultrasound system located outside of the sterile field, without the operator leaving the sterile field of surgery. Specifically, the inclusion of a smart button on the probe facilitates the operator's command of the ultrasound system.

The smart button may be available within the sterile field, and may be embodied as a button on the probe, a foot-switch, a voice-activated input, etc. Pressing the button while pointing the tracked external device towards the imaging field of the tracked ultrasound probe facilitates saving of the intersection coordinates between the instruments. This may be used to highlight, or pin-down anatomical landmarks found within the ultrasound window, without having to interact with the physical ultrasound system, and therefore without leaving the sterile field of surgery.

Technically and beneficially, the operator can highlight anatomical structures of interest in the ultrasound image without having to leave the sterile field of surgery. This minimizes a risk of contaminating the sterile field, while also permitting improved ergonomics during the procedure, as the operator can interact with the ultrasound system without the system having to be within reach of the operator and without the operator having to move around the operating room.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

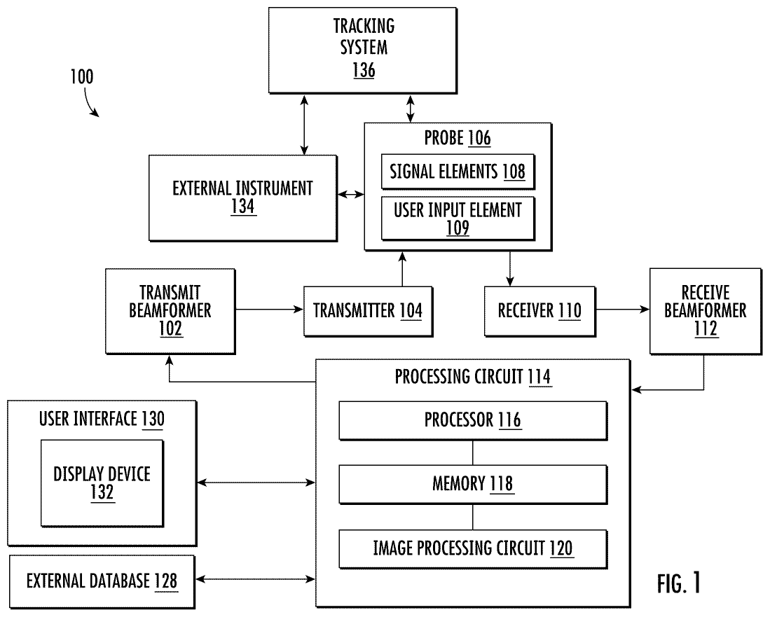

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient. For example, as will be described herein, the sonographer may be a surgeon performing a surgery on a patient that requires the use of ultrasound images to guide the surgery. Although the systems and methods are described herein in the context of the ultrasound imaging system 100, it should be appreciated that the ultrasound tracking and smart button described herein may be implemented using any of a variety of medical imaging systems (e.g., medical resonance imaging, x-ray, computed tomography, positron emission tomography, etc.).

An example of a procedure performed using the ultrasound imaging system 100 may be an intraoperative ultrasound, or an ultrasound performed during surgery. In some embodiments, an intraoperative ultrasound may permit a transducer of an ultrasound probe to be placed directly on the organ or anatomy to be imaged. As an example, an intraoperative ultrasound may be a liver ultrasound that is performed during a surgery on a patient's liver. The ultrasound may be performed to identify lesions on the liver. During the liver ultrasound, the surgeon may move the probe across the liver or a tissue surrounding the liver (e.g., across skin above the liver) and identify lesions, such as tumors, hemangiomas, cysts, etc.

In at least one embodiment, the sonographer collects the ultrasound data by navigating a probe (e.g., probe 106, as described below) over the patient's anatomical structure of interest (e.g., liver) until a sufficient volume of ultrasound images are collected. The collected images are stored in a central storage device (e.g., memory 118) and analyzed by the sonographer. The sonographer generates a set of measurements from the images (e.g., 50-100 records), and the images and measurements are collectively reviewed by a physician (e.g., the surgeon performing the ultrasound). The surgeon uses the images to guide the surgery and provides any clinical findings/conclusions in a report submitted to the patient's medical record.

As shown in FIG. 1, the ultrasound imaging system 100 includes a transmit beamformer 102, a transmitter 104, a probe 106, a receiver 110, a receive beamformer 112, an external instrument 134, and a tracking system 136.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor or marker configured to detect a position of the probe 106 relative to one or more reference locations. That is, the position sensor or marker may continuously track movement (e.g., rotation, translation, orientation, etc.) of the probe 106 relative to the location of the probe 106 when the anatomy being imaged is identified. For example, the anatomy being imaged may be identified as a liver at a first location of the probe 106. Then, the position sensor or marker may track the movement of the probe 106 relative to the liver in order to identify successive locations of the probe 106. In some embodiments, the position sensor or marker may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during a liver ultrasound, a sonographer or other clinician may navigate the probe 106 proximate to a patient's liver so that the signal elements 108 in the probe 106 emit the pulsed ultrasonic signals into the patient's liver. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the signal elements 108), and a damping block configured to absorb ultrasound energy.

The probe 106 may also include a user input element 109. The user input element 109 may be a smart button or other component (e.g., foot pedal, etc.) that can receive an input from the sonographer to control the ultrasound imaging system. For example, the user input element 109 may be a button or other element that permits the sonographer to control a display characteristic of the displayed ultrasound image and/or an operating characteristic of the transducer of the probe.

The probe 106 and/or user input element 109 may include one or more sensors or markers to track a location of the probe 106 within space. For example, the probe 106 may include one or more external sensors and/or may have an integrated tracking system. As will be discussed in greater detail herein, the probe 106 may be tracked using spatial tracking, such as electromagnetic (EM) tracking, optical tracking, etc. Tracking the probe 106 may indicate what tissue or tissues, anatomical structures, etc. are being imaged. Specifically, tracking the probe 106 may indicate a position of the probe 106 relative to other objects in space.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, and an image processing circuit 120. In this way, the processing circuit 114 may be structured or configured to execute or implement the instructions, commands, and/or control processes described herein with respect to the processor 116, the memory 118, and the image processing circuit 120. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to some embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using user interface 130 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-2 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Similarly, when the ultrasound imaging system 100 obtains a relatively large volume of data, the real-time volume-rate may be slower than for a smaller volume of data.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

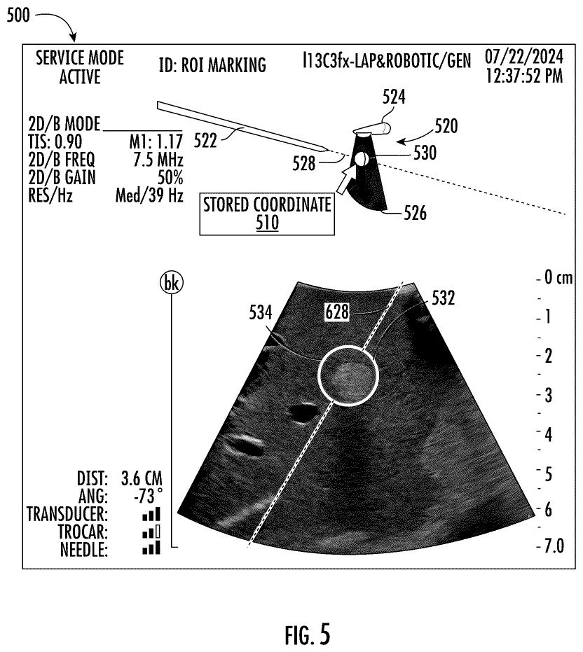

The processor 116 may also be in electronic communication with the display device 132 such that the processor 116 may process ultrasound data obtained by the probe 106 and generate images to display on the display device 132 (e.g., ultrasound images 500, 600, and 700, as described below with reference to FIGS. 5-7).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120. The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during a liver ultrasound and may therefore include various images of a patient's liver and the structures contained therein. The image processing circuit 120 may include multiple deep learning-based models configured to analyze the image data. For example, the image processing circuit may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

The ultrasound imaging system 100 may also include an external database 128 and a user interface 130. The external database 128 refers to a database from which the processing circuit 114 (e.g., image processing circuit 120) may retrieve information used to provide recommended imaging parameters during an ultrasound scan. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, image processing circuit 120 may retrieve clinical guidelines, standard practices, medical literature, medical textbooks, published research, and previous case studies related to the implementation and/or procedure. For example, if the ultrasound imaging system 100 is being used in a hospital setting to perform a first trimester fetal ultrasound on a patient with a BMI of 28.0, image processing circuit 120 may retrieve clinical guidelines and standard practices related to the hospital setting and the first trimester fetal ultrasound. Continuing with this example, image processing circuit 120 may also retrieve information from the medical literature, medical textbooks, published research, and previous case studies related to fetal anatomy and the risks associated with having a high BMI during pregnancy.

The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. In some embodiments, the user interface 130 may be an electronic device that was designed and developed for use in a medical environment. As described above, the user input element 109 may be used in addition to or alternative to the user interface 130 to control certain aspects of operation of the ultrasound imaging system 100.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

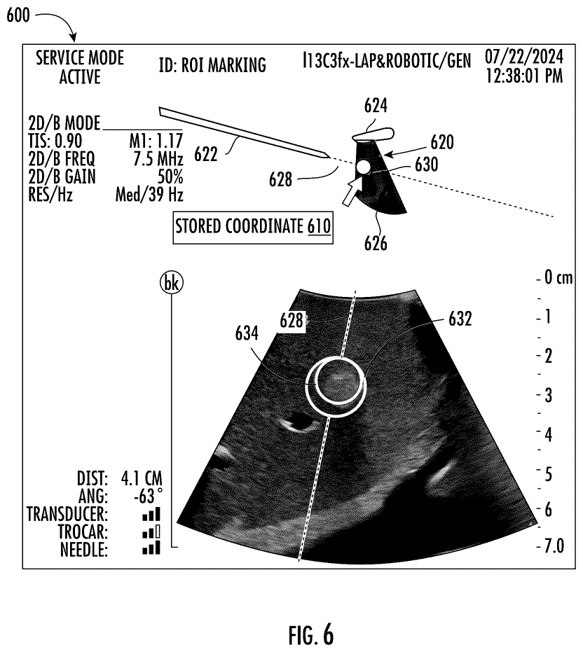

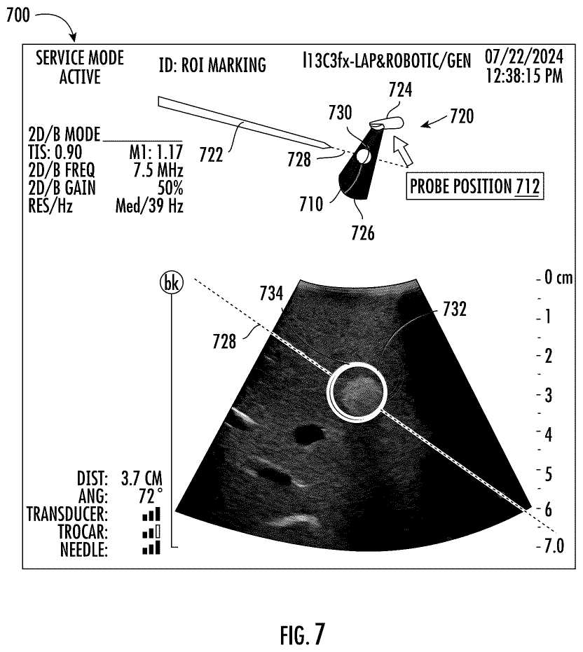

The ultrasound imaging system 100 may also include an external instrument 134. The external instrument 134 may be any instrument used by the sonographer during surgery and/or during ultrasound imaging. The external instrument 134 may be, for example, a needle, a stylus, a trocar, and the like. In various embodiments, the external instrument 134 may include one or more sensors or markers to track a location of the external instrument 134 in space. Specifically, the location of the external instrument 134 relative to the tissue being imaged (and, therefore, relative to the location of the probe 106) may be tracked. In various embodiments, the location of the external instrument 134 may be tracked using one or more external sensors or markers, an integrated tracking system, EM tracking, etc. As will be shown in FIGS. 6 and 7, a representation of the external instrument 134, or a representation based on the external instrument 134 (e.g., such as a trajectory of the external instrument 134), may be displayed on the display device 132 within a generated ultrasound image.

The ultrasound imaging system 100 also includes a tracking system 136. Specifically, in some embodiments, the tracking system is a spatial tracking system 136. The tracking system 136 may enable tracking of the probe 106 and/or the external instrument 134. The tracking system 136 may be external to the ultrasound imaging system 100 or integrated within the ultrasound imaging system 100. The tracking system 136 may include a stationary signal generator and/or receiver and one or more sensors or markers placed on the probe 106 and/or the external instrument 134. As described above, the probe 106 and the external instrument 134 may include one or more sensors or markers that enable tracking of the same and with respect to one another. The locations of the probe 106 and the external instrument 134 may be expressed as coordinates (e.g., x, y, z coordinates) and may include an orientation.

In one embodiment, the tracking system 136 is an electromagnetic tracking system. In such embodiments, the tracking system 136 may be or include an electromagnetic (EM) field generator. The EM field generator may be configured to generate an electromagnetic field to enable EM tracking of the probe 106 and the external instrument 134. For example, the EM field generator may be an external magnet placed proximate an imaging location on the patient when an ultrasound image is to be obtained, and the probe 106 and the external instrument 134 may include one or more sensors that enable EM tracking of the same and with respect to one another. The sensors may therefore detect the generated EM field and determine a location of the sensors (and, therefore the probe/instrument) within the magnetic field.

In another embodiment, the tracking system is an optical tracking system. In such embodiments, the tracking system 136 may be or include a camera, one or more light-emitting sources, and one or more markers. The probe 106 and the external instrument 134 may have markers attached, which may be markers that reflect light from the external source and/or markers that emit light. The cameras may track the positions of the markers, and the tracking system 136 may determine the positions of the probe 106 and the external instrument using the markers and known (e.g., stationary) positions of the cameras.

The tracking system 136 may be or include any other types of spatial tracking systems, such as RFID tracking systems, inertial measurement unit (IMU) tracking systems, etc.

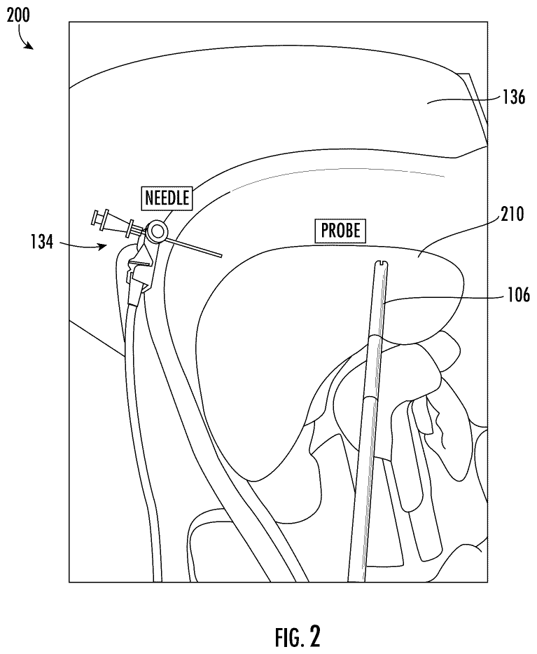

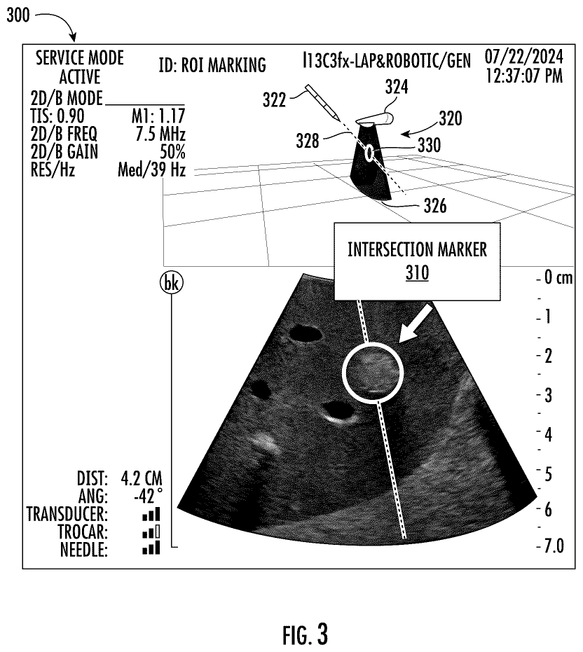

Referring now to FIG. 2, a diagram 200 showing an ultrasound probe 106 and an external instrument 134 is shown, according to an example embodiment. As shown in the diagram 200, the external instrument 134 is embodied as a needle. The diagram 200 depicts a system using spatial tracking. As shown, the tracking system 136 (e.g., an EM field generator, an optical tracking system, etc.) is positioned proximate the tissue being imaged, shown as liver 210. The probe 106 is positioned on the liver 210 to obtain an image of the liver 210. The needle 134 is positioned proximate the liver 210 and the probe 106. Both the needle 134 and the probe 106 include sensors (e.g., EM sensors, optical markers, etc.) that interact with the spatial tracking system 136. For example, EM sensors may interact with a magnetic field generated by the EM field generator. As another example, optical markers may interact with one or more cameras. Thus, as will be shown in FIG. 3, as the needle 134 and the probe 106 move, coordinates of the needle 134 and the probe 106 may be stored and a representation of the needle 134 may be generated and displayed on the ultrasound image to show a position of the needle 134 and the probe 106 (e.g., the portion of the liver 210 being imaged) relative to each other and the tracking system 136.

Referring now to FIG. 3, an illustration of an ultrasound image 300 showing an intersection between an ultrasound probe and an external instrument is shown, according to an example embodiment. The ultrasound image 300 corresponds to the embodiment depicted in FIG. 2. As shown, the ultrasound image 300 includes an intersection marker 310, showing an intersection point between the needle 134 and the probe 106. The ultrasound image 300 also includes a representation 320 of the needle 134 and the probe 106. The representation 320 includes a needle representation 322 and a probe representation 324, each indicating a position and orientation of each in space. The representation 320 further includes an imaging plane representation 326, indicating an orientation and position of the current imaging plane. Additionally, the needle representation 322 includes a projected trajectory 328, indicating a projected trajectory of the needle 134 if the needle 134 continued moving along a current trajectory associated with its current position and orientation. The representation 320 further includes an intersection 330 that indicates a point of intersection or projected point of intersection between the needle 134 and the tissue being imaged. The intersection 330 may indicate the same location and/or position that is indicated by the intersection marker 310, but shown in a different orientation in space.

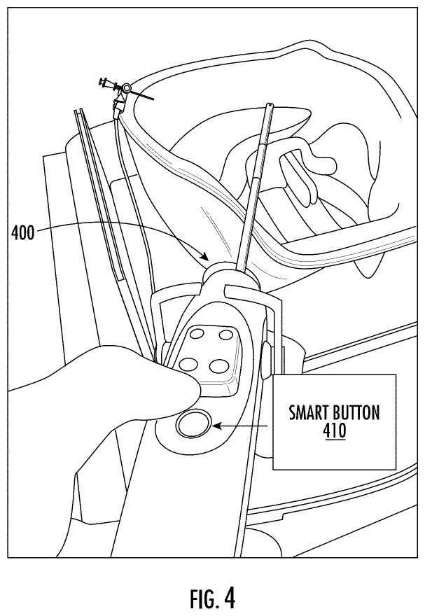

Referring now to FIG. 4, an illustration of an ultrasound probe 400 including a user input element is shown, according to an example embodiment. The ultrasound probe 400 may be the same as or similar to the probe 106 described with respect to FIG. 1. As shown, the probe 400 may include a user input element 410, shown in FIG. 4 as a smart button 410. The smart button 410 may be the same as the user input element 109 described with respect to FIG. 1.

The user input element 410 may be a smart button or other component that can receive an input from the sonographer to control the ultrasound imaging system. For example, the user input element 410 may be a button on the probe 400 that can be actuated or pressed by the sonographer, a foot pedal coupled with the probe 400 or other component(s) of the ultrasound imaging system, a remote control, etc. that permits the sonographer to control a display characteristic of the displayed ultrasound image and/or an operating characteristic of the transducer of the probe. In some embodiments, the user input element 410 may be a voice-activated feature integrated with the probe 400. For example, the probe 400 may include a speaker that can receive and identify a voice command to control the ultrasound system in some way (e.g., a display characteristic of the displayed image and/or an operating characteristic of the transducer).

In various embodiments, controlling the display characteristic of a displayed ultrasound image may include marking a point of intersection between a tissue imaged to be imaged (hereinafter “tissue”) by the probe 400 and the external instrument 134, freezing the displayed image, capturing the displayed image, storing the displayed image, modifying a depth of the displayed image, modifying a frequency of the displayed image, etc. Controlling an operating characteristic of the transducer may include modifying an imaging depth of the transducer, an imaging or acquisition frequency of the transducer, changing between modes of operation, etc. In some embodiments, interacting with the user input element 410 may cause a menu to be displayed on a display device (e.g., the display device 132 that will be described herein). For example, the user input element 410 may be embodied as a button on the probe 400, and pressing the button may cause a menu showing a plurality of different options for controlling the display device and/or the transducer. For example, the sonographer may press the button and options for freezing the displayed image or saving the displayed image may be displayed. Options for modifying an imaging depth and an imaging frequency may also be displayed.

As such, in some embodiments, the user input element 410 may be a plurality of elements to navigate the menu. For example, the user input element 410 may include a first button that can select different options, and a plurality of second buttons that can move to different options (e.g., a plurality of arrows to navigate around the display device 132). In embodiments where the user input element 410 is a foot pedal, for example, the user input element 410 may include a plurality of pedals, each pedal corresponding to a different operation (e.g., a pedal to select an option and a plurality of pedals to move amongst different options). In some embodiments, the external instrument 134 may act as a mouse or cursor to select an option on the displayed menu. For example, through EM tracking or any other type of spatial tracking, the sonographer or healthcare professional may move the external instrument 134, and a representation of the external instrument shown on the generated ultrasound image may move on the display screen to point to different menu options that may be selected by, for example, pressing the smart button.

In some embodiments, the user input element 410 may include a single element (e.g., a single button, pedal, etc.). Different numbers of successive actuations of the user input element 410 may cause different aspects of the imaging system to be controlled. For example, actuating the user input element 410 once (e.g., pushing the button or pedal once) may cause the displayed image to be frozen. Actuating the user input element 410 twice in succession (e.g., “double-clicking” the button or pedal) may cause the displayed image to be stored. In embodiments where the user input element 410 is a voice command system, the sonographer may speak key words or commands (e.g., “freeze,” “freeze this image,” “store,” “store this image,” “increase imaging depth,” “decrease imaging depth,” etc.) that cause the displayed image and/or transducer to be controlled. In some embodiments, the user input element 410 may be activated via one or more gestures. For example, the ultrasound imaging system 100 may include a camera or other facial recognition software. The sonographer may gesture (e.g., blink, wink, etc.), and each gesture may correspond to a different control (e.g., blinking once freezes an image, blinking twice increases an imaging depth, smiling stores an image, etc.). The camera may recognize the gesture and correspondingly control the ultrasound system. In some embodiments, optical tracking may be implemented (e.g., a camera identifies different eye movements, such as moving eyes up and down or left and right, to control the ultrasound system).

Additionally, in some embodiments, the probe 106 and/or another instrument or device may be moved to control the ultrasound system. For example, a device may be kept stationary for greater than or equal to a predetermined amount of time. The device may then be moved (e.g., wiggled, shaken, etc.), and the imaging system may be appropriately controlled. For example, an instrument may be held still for 10 seconds, and, after at least 10 seconds, the instrument may be shaken to freeze an ultrasound image being displayed.

As described herein, the probe 400 and/or user input element 410 may include one or more sensors or markers to track a location of the probe 400 within space. For example, the probe 400 may include one or more sensors to facilitate spatial tracking of the probe 400.

Referring now to FIG. 5, an illustration of an ultrasound image 500 showing a stored coordinate is shown, according to an example embodiment. Specifically, the ultrasound image 500 includes a representation 520 of the spatially tracked ultrasound system. The representation 520 may be similar to the representation 320 described with respect to FIG. 3. That is, the representation 520 includes an external instrument representation 522, a probe representation 524, an imaging plane 526, an external instrument trajectory 528, and an intersection point 530, each of which may be similar to the respective elements 322-330 described with respect to FIG. 3.

The representation 520 further includes a stored coordinate 510. The store coordinate may be generated by actuating a user input element (e.g., the user input element 109 or 410). For example, the sonographer may align the external instrument 134 such that the external instrument trajectory 528 shows that the external instrument 134 is to intersect with the tissue being imaged at an intersection point 530. Upon identifying the intersection point (and/or moving the external instrument 134 and/or the probe 106) such that the external instrument 134 intersects with the tissue, the sonographer may actuate the user input element 109 to store the intersection point. For example, the sonographer may push a smart button, push a foot pedal, use a voice command to indicate that an intersection point should be stored, etc. The stored coordinate 510 may be a coordinate determined or identified based on the coordinates and positions of the external instrument 134 and the probe 106 identified using spatial tracking, such as EM or optical tracking. It should be understood that the user input element 109 may be actuated to perform any number of operations and is not limited to storing coordinates. For example, upon identifying an intersection between the external instrument and the tissue being imaged, the user input element 109 may be actuated to store a coordinate, freeze the image at while the instrument intersects the tissue, store the image, modify one or more imaging parameters, etc.

The ultrasound image 500 may also include the intersection point 532 and the stored coordinate 534. The intersection point 532 and the stored coordinate 534 may be the same as the intersection point 530 and the stored coordinate 510 but shown in a different plane or point of view.

In various embodiments, the ultrasound imaging system 100 may be configured to store multiple coordinates and intersection points. For example, the external instrument may be configured as needle. During the ultrasound, the sonographer may position the probe 106 and the needle such that the needle intersects with the tissue being imaged. The sonographer may store the coordinate and subsequently remove the needle from that position. Further, the probe 106 may be moved to obtain an ultrasound image of another portion of the tissue or anatomical structure. The sonographer may then reinsert the needle such that the needle and intersects with the new portion of tissue that is being imaged, and the coordinates of intersection may be stored. This may allow the sonographer to view multiple stored coordinates. This may be beneficial in aiding the sonographer in distinguishing between anatomical structures that have already been viewed and those that have not. For example, a sonographer may be imaging a liver containing multiple lesions. The sonographer may mark a point of intersection between the external element and each identified lesion. Storing the coordinates of each intersection may allow the sonographer to easily identify which lesions have been identified and imaged and which lesions have not, thereby reducing a likelihood that a lesion is missed or the same lesion is identified multiple times.

Referring now to FIG. 6, an illustration of an ultrasound image 600 showing a stored coordinate is shown, according to another example embodiment. Specifically, the ultrasound image 600 includes a representation 620 of the spatially tracked ultrasound system. The representation 620 may be similar to the representation 520 described with respect to FIG. 5. That is, the representation 620 includes an external instrument representation 622, a probe representation 624, an imaging plane 626, an external instrument trajectory 628, an intersection point 630 and a stored coordinate 610, each of which may be similar to the respective elements 610-630 described with respect to FIG. 5.

The representation 620 shows a change in orientation of the imaging plane 626 relative to the imaging plane 526 shown and described in FIG. 5. For example, the probe 106 may be positioned at a different angle than as shown in FIG. 5, thereby adjusting a position of the imaging plane 626 and the intersection point 630 relative to those in FIG. 5. In various embodiments, the sonographer may mark a stored coordinate 610 that does not completely align with the intersection point 630. As such, the intersection marker 632 and the stored coordinate marker 634 may not completely align (e.g., as they do in FIG. 5 when the intersection point 530 and the stored coordinate 510 completely align).

FIG. 7 is an illustration of an ultrasound image showing a position of an ultrasound probe, according to an example embodiment. Specifically, the ultrasound image 700 includes a representation 720 of the spatially tracked ultrasound system. The representation 720 may be similar to the representations 520 and 620 described with respect to FIGS. 5 and 6, respectively. That is, the representation 720 includes an external instrument representation 722, a probe representation 724, an imaging plane 726, an external instrument trajectory 728, an intersection point 730 and a stored coordinate 710, each of which may be similar to the respective elements 610-630 described with respect to FIG. 6 or the elements 510-530 described with respect to FIG. 5.

The representation 720 shows a different probe position 712 relative to the probe positions shown in FIGS. 5 and 6. For example, the probe representation 724 shows that the probe 106 is positioned at a different angle and/or orientation relative to the probe positions shown in FIGS. 5 and 6. This is also shown by the imaging plane 726 being positioned at a different angle relative to the imaging planes 526 and 626.

As shown in FIG. 7, the stored coordinate 710 may be a coordinate corresponding to the intersection point 730 between the needle representation 722 and the tissue. Accordingly, the intersection marker 732 and the stored coordinate 734 are shown to align or substantially align (e.g., the representations are concentric or near-concentric. Additionally, due to the altered probe position 712, the trajectory 728 may be positioned differently relative to the positions of the trajectories 528 and 628.

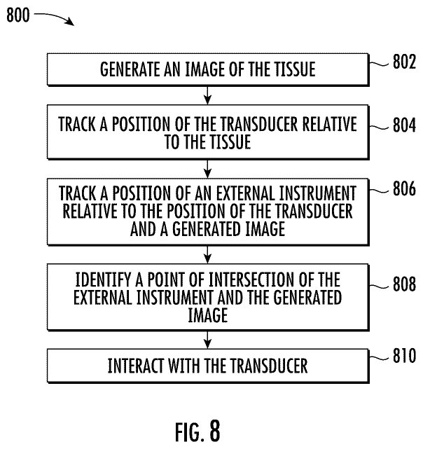

Referring now to FIG. 8, a flow diagram illustrating a method 800 of controlling an ultrasound system using spatial tracking is shown, according to an example embodiment. The processes described herein may be performed by the processing circuit 114 and/or one or more components of the ultrasound imaging system 100.

At process 802, an image of a tissue is generated. For example, a probe (e.g., the probe 106) may be moved along a tissue to image an anatomical structure, and an image of the tissue may be generated and displayed (e.g., on the display device 132).

At process 804, a position of a transducer of the probe is tracked. The position of the transducer is tracked relative to the tissue. The transducer may be tracked using spatial tracking facilitated by, for example, the generation of an electromagnetic field or light emission, and sensors or markers included on the probe. The position of the transducer may be tracked based on the probe's position relative to the tracking system 136. Specifically, the position of the transducer may be tracked based on the probe's position relative to a stationary component of the tracking system 136. For example, in embodiments where spatial tracking is performed using EM tracking, the position of the transducer may be tracked based on the probe's position relative to an EM field generator.

At process 806, a position of an external instrument (e.g., the external instrument 134) is tracked. The external instrument may be a needle, a trocar, a stylus, etc. The position of the external instrument is tracked relative to the position of the transducer and the generated image of the tissue. The transducer may be tracked using spatial tracking facilitated by, for example, the generation of an electromagnetic field or emission of light, and sensors or markers included on the external instrument. The position of the external instrument may be tracked based on the instrument's position relative to the tracking system 136 (e.g., a stationary component of the tracking system 136, such as an EM field generator). The tracked position of the external instrument may indicate a calculated trajectory of the external instrument, and the point of intersection between the instrument and the tissue may be a predicted point of intersection between the instrument and the tissue.

At process 808, a point of intersection of the external instrument and the generated image is identified. For example, as the probe and/or external instrument are moved, the generated image, based on spatial tracking, may identify a point of intersection or projected point of intersection between the external instrument and the tissue being imaged.

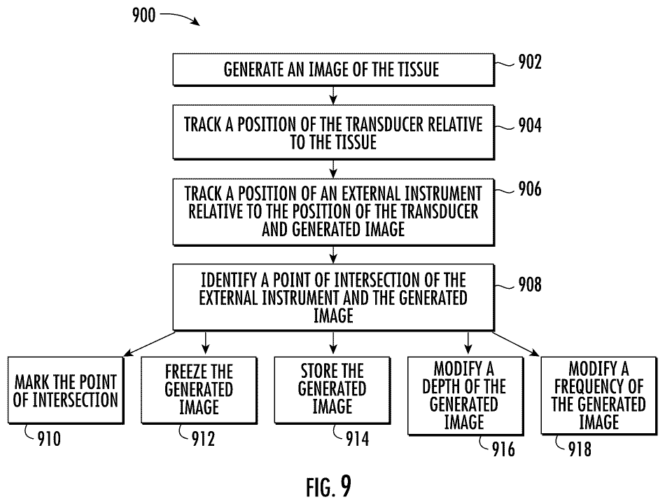

At process 810, the transducer receives an interaction (e.g., a user input) and the system receives an indication of the interaction. For example, a user may interact with a user input element (e.g., the user input element 109) on the probe. The user input element may be, for example, a smart button on the probe, a foot pedal, a remote control, a camera for identifying a body gesture, a microphone for receiving a voice command, etc. The interaction may be received, and a display characteristic of the displayed image or an operating characteristic of the transducer may be controlled. Examples of an interaction may include, for example, pressing a smart button a certain number of times, pressing a foot pedal a certain number of times, initiating a voice command, gesturing, etc. Examples of these characteristics being controlled are described in greater detail with respect to FIG. 9.

Referring now to FIG. 9, a flow diagram illustrating a method 900 of controlling an ultrasound system using spatial tracking is shown, according to another example embodiment. The processes 902-908 may be the same as or similar to processes 802-808 described above with respect to FIG. 8. The processes described herein may be performed by the processing circuit 114 and/or one or more components of the ultrasound imaging system 100.

At process 902, an image of a tissue is generated. At process 904, a position of a transducer of a probe is tracked. The position of the transducer is tracked relative to the tissue. At process 906, a position of an external instrument is tracked. The position of the external instrument is tracked relative to the position of the transducer and the generated image of the tissue. At process 908, a point of intersection of the external instrument and the generated image is identified.

Responsive to identifying the point of intersection, the ultrasound display image and/or the transducer may be controlled. For example, one or more of the processes 910-916 described below may occur. The processes 910-916 may occur responsive to receipt of an interaction, by a user, with a user input element of the probe. For example, a user may interact with a user input element (e.g., the user input element 109) on the probe. The user input element may be, for example, a smart button on the probe, a foot pedal, a remote control, a camera for identifying a body gesture, a microphone for receiving a voice command, etc. In some embodiments, each process 910-916 may occur responsive to a different type of interaction with the user input element.

At process 910, the point of intersection identified at process 908 is marked. For example, responsive to a user input to a user input element on the probe, the point of intersection is marked. The point of intersection may be marked as a stored coordinate. As the probe and external element move around the tissue being imaged, a plurality of points of intersection may be marked and stored as coordinates.

At process 912, the generated image is frozen. For example, responsive to a user input to a user input element on the probe, the generated and displayed ultrasound image is frozen and/or captured.

At process 914, the generated image is stored. For example, responsive to a user input to a user input element on the probe, the generated and displayed ultrasound image is stored (e.g., in the memory 118). The image may be stored for future use.

At process 916, a depth of the generated image is modified. For example, responsive to a user input to a user input element on the probe, an imaging parameter of the probe may be modified. For example, the transducer of the probe may adjust an imaging depth (e.g., increase or decrease an imaging depth), thereby causing the depth of the generated image to be modified.

At process 918, a frequency of the generated image is modified. For example, responsive to a user input to a user input element on the probe, an imaging parameter of the probe may be modified. For example, the transducer of the probe may adjust an imaging frequency (e.g., increase or decrease a frequency or image acquisition rate), thereby causing the appearance of the generated image to be modified.

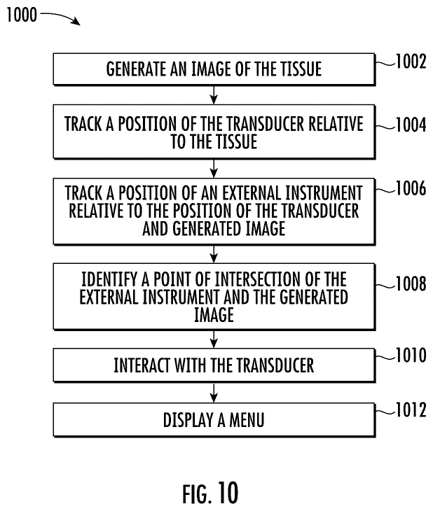

Referring now to FIG. 10, a flow diagram illustrating a method 1000 of controlling an ultrasound system using spatial tracking is shown, according to another example embodiment. The processes 1002-1010 may be the same as or similar to processes 802-810 described above with respect to FIG. 8. The processes described herein may be performed by the processing circuit 114 and/or one or more components of the ultrasound imaging system 100.

At process 1002, an image of a tissue is generated. At process 1004, a position of a transducer of a probe is tracked. The position of the transducer is tracked relative to the tissue. At process 1006, a position of an external instrument is tracked. The position of the external instrument is tracked relative to the position of the transducer and the generated image of the tissue. At process 1008, a point of intersection of the external instrument and the generated image is identified. At process 1010, the transducer receives an interaction (e.g., a user input) and the system receives an indication of the interaction.

At process 1012, a menu is displayed on the generated and displayed ultrasound image. For example, a menu may be overlaid on the ultrasound image. The menu may be displayed responsive to receipt of the indication of the user interaction with the transducer. The menu may include a plurality of options including at least one of: marking the point of intersection, freezing the generated image, storing the generated image, modifying a depth of the generated image, and/or modifying a frequency of the generated image. The user input element and/or the external instrument may be actuated to select at least one of the plurality of elements.

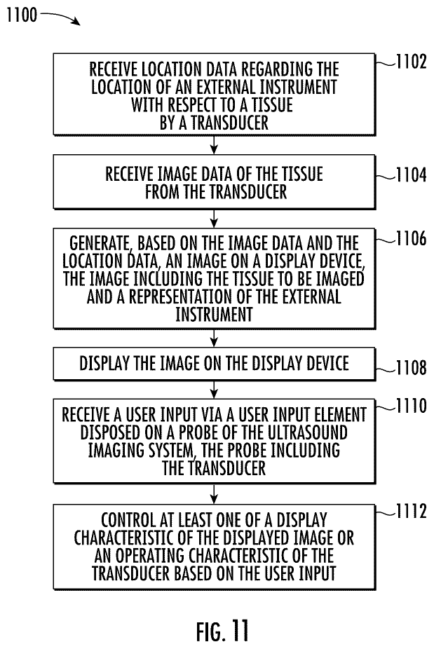

FIG. 11 is a flow diagram illustrating a method 1100 of controlling an ultrasound system using spatial tracking, according to another example embodiment. The processes described herein may be performed by the processing circuit 114 and/or one or more components of the ultrasound imaging system 100.

At process 1102, location data is received regarding a location of an external instrument (e.g., the external instrument 134). The location of the external instrument may be with respect to a tissue imaged by a transducer of a probe (e.g., the probe 106). In some embodiments, the external instrument is at least one of a needle, a stylus, or a trocar.

At process 1104, image data of the tissue is received from the transducer.

At process 1106, an image is generated based on the location data received at process 1102 and the image data received at process 1104. The image may include the tissue and a representation of the external instrument. The representation of the external instrument may be generated based on spatial tracking of the external instrument.

At process 1108, the image generated at process 1106 is displayed. The image may be displayed on a display device (e.g., the display device 132) of the ultrasound imaging system 100.

In some embodiments, the image includes a point of intersection between the representation of the external instrument and the tissue. As such, in some embodiments, the method 1100 further includes marking the point of intersection by storing coordinates of the point of intersection. The coordinates may be relative to a stationary position of a spatial tracking system. For example, the coordinates may be relative to a stationary component of the spatial tracking system, such as an EM field generator when EM tracking is used or a camera when optical tracking is used.

At process 1110, a user input is received via a user input element (e.g., the user input element 109). The user input element may be disposed on a probe (e.g., the probe 106) of the ultrasound imaging system 100.

At process 1112, at least one of a display characteristic of the displayed image or an operating characteristic of the transducer is controlled. The display characteristic and/or the operating characteristic may be controlled based on the user input received at process 1110.

In some embodiments, controlling the at least one of the display characteristic of the displayed image or the operating characteristic of the transducer includes at least one of: marking a point of intersection between the representation of the external instrument and the tissue, freezing the displayed image, storing the displayed image, modifying a depth of the displayed image, or modifying a frequency of the displayed image.

In some embodiments, the user input element includes one or more of: a button, a footswitch, or a remote control. In some embodiments, the user input includes actuating one or more of the button, the footswitch, or the remote control to control the at least one of the display characteristic of the displayed image or the operating characteristic of the transducer to: mark a point of intersection between the representation of the external instrument and the tissue, freeze the displayed image, store the displayed image, modify a depth of the displayed image, or modify a frequency of the displayed image.

In some embodiments, the user input element is the button. Further, in some embodiments, the button is voice activated. Thus, the received user input may include a voice command to control the at least one of the display characteristic of the displayed image or the operating characteristic of the transducer to: mark the point of intersection between the representation of the external instrument and the tissue, freeze the displayed image, store the displayed image, modify the depth of the displayed image, or modify the frequency of the displayed image.

In some embodiments, the received user input includes a shaking of the user input element to control the at least one of the display characteristic of the displayed image or the operating characteristic of the transducer to: mark a point of intersection between the representation of the external instrument and the tissue, freeze the displayed image, store the displayed image, modify a depth of the displayed image, or modify a frequency of the displayed image.

In some embodiments, controlling at least one of the display characteristic of the displayed image or the operating characteristic of the transducer causes a menu to be displayed on the display device. The menu may include a plurality of options. The plurality of options may include at least one of: marking a point of intersection between the representation of the external instrument and the tissue, freezing the generated image, storing the generated image, modifying a depth of the generated image, or modifying a frequency of the generated image. In some embodiments, the user input element and/or the external instrument is actuated to select at least one of the plurality of options.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In some embodiments, the volatile storage media may take the form of RAM, TRAM, ZRAM, etc. Combinations of the above are also included within the scope of machine-readable media. In this regard, machine-Atty. executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

1. An ultrasound imaging system comprising:
a transducer configured to transmit and receive an ultrasound signal;
a matching layer configured to have an acoustic impedance between a tissue to be imaged and a material of the transducer;
a damping block configured to absorb ultrasound energy;
an external instrument;
a spatial tracking system configured to enable tracking of the transducer and the external instrument; and
a processing circuit comprising a processor coupled to a memory device storing instructions thereon that, when executed, cause the processing circuit to perform operations comprising:
receiving location data regarding the location of the external instrument with respect to a tissue imaged by the transducer;
receiving image data of the tissue from the transducer;
generating and displaying, based on the image data and the location data, an image on a display device of the ultrasound imaging system, the image comprising the tissue and a representation of the external instrument;
receiving a user input via a user input element disposed on [a] an ultrasound imaging probe of the ultrasound imaging system, the probe comprising the transducer; and
controlling at least one of a display characteristic of the displayed image or an operating characteristic of the transducer based on the user input.

2. The ultrasound imaging system of claim 1, wherein controlling the at least one of the display characteristic of the displayed image or the operating characteristic of the transducer comprises at least one of: marking a point of intersection between the representation of the external instrument and the tissue, freezing the displayed image, storing the displayed image, modifying a depth of the displayed image, or modifying a frequency of the displayed image.

3. The ultrasound imaging system of claim 1, wherein the user input element comprises one or more of: a button, a footswitch, or a remote control, and wherein the user input comprises actuating one or more of the button, the footswitch, or the remote control to control the at least one of the display characteristic of the displayed image or the operating characteristic of the transducer to: mark a point of intersection between the representation of the external instrument and the tissue, freeze the displayed image, store the displayed image, modify a depth of the displayed image, or modify a frequency of the displayed image.

4. The ultrasound imaging system of claim 1, wherein the user input element comprises a microphone, and wherein the microphone is voice activated, and wherein the received user input comprises a voice command to control the at least one of the display characteristic of the displayed image or the operating characteristic of the transducer to: mark the point of intersection between the representation of the external instrument and the tissue, freeze the displayed image, store the displayed image, modify the depth of the displayed image, or modify the frequency of the displayed image.

5. The ultrasound imaging system of claim 1, wherein the received user input comprises a shaking of the user input element to control the at least one of the display characteristic of the displayed image or the operating characteristic of the transducer to: mark a point of intersection between the representation of the external instrument and the tissue, freeze the displayed image, store the displayed image, modify a depth of the displayed image, or modify a frequency of the displayed image.

6. The ultrasound imaging system of claim 1, wherein the external instrument is at least one of a needle or a trocar.

7. The ultrasound imaging system of claim 1, wherein the image comprises a point of intersection between the representation of the external instrument and the tissue, wherein the operations further comprise:
marking the point of intersection by storing coordinates of the point of intersection, the coordinates relative to a stationary position of the spatial tracking system.

8. The ultrasound imaging system of claim 1, wherein controlling at least one of the display characteristic of the displayed image or the operating characteristic of the transducer causes a menu to be displayed on the display device, the menu comprising a plurality of options, the plurality of options comprising at least one of: marking a point of intersection between the representation of the external instrument and the tissue, freezing the generated image, storing the generated image, modifying a depth of the generated image, or modifying a frequency of the generated image.

9. The ultrasound imaging system of claim 8, wherein the user input element is configured to be actuated to select at least one of the plurality of options.

10. The ultrasound imaging system of claim 1, wherein the representation of the external instrument is generated based on the tracking of the external instrument.

11. A medical imaging system comprising:
a processing circuit comprising a processor coupled to a memory device storing instructions thereon that, when executed, cause the processing circuit to perform operations comprising:
receiving location data regarding the location of an external instrument with respect to a tissue imaged by a transducer;
receiving image data of the tissue from the transducer;
generating and displaying, based on the image data and the location data, an image on a display device of the medical imaging system, the image comprising the tissue and a representation of the external instrument;
receiving a user input via a user input element disposed on an ultrasound imaging probe of the medical imaging system, the probe comprising the transducer; and
controlling at least one of a display characteristic of the displayed image or an operating characteristic of the transducer based on the user input.

12. The medical imaging system of claim 11, wherein controlling the at least one of the display characteristic of the displayed image or the operating characteristic of the transducer comprises at least one of: marking a point of intersection between the representation of the external instrument and the tissue, freezing the displayed image, storing the displayed image, modifying a depth of the displayed image, or modifying a frequency of the displayed image.

13. The medical imaging system of claim 11, wherein the user input element comprises one or more of: a button, a footswitch, or a remote control, and wherein the user input comprises actuating one or more of the button, the footswitch, or the remote control to control the at least one of the display characteristic of the displayed image or the operating characteristic of the transducer to: mark a point of intersection between the representation of the external instrument and the tissue, freeze the displayed image, store the displayed image, modify a depth of the displayed image, or modify a frequency of the displayed image.

14. The medical imaging system of claim 13, wherein the user input element comprises a microphone, and wherein the microphone is voice activated, and wherein the received user input comprises a voice command to control the at least one of the display characteristic of the displayed image or the operating characteristic of the transducer to: mark the point of intersection between the representation of the external instrument and the tissue, freeze the displayed image, store the displayed image, modify the depth of the displayed image, or modify the frequency of the displayed image.

15. The medical imaging system of claim 11, wherein the image comprises a point of intersection between the representation of the external instrument and the tissue, wherein the operations further comprise:
marking the point of intersection by storing coordinates of the point of intersection, the coordinates relative to a stationary position of a spatial tracking system.

16. The medical imaging system of claim 11, wherein controlling at least one of the display characteristic of the displayed image or the operating characteristic of the transducer causes a menu to be displayed on the display device, the menu comprising a plurality of options, the plurality of options comprising at least one of: marking a point of intersection between the representation of the external instrument and the tissue, freezing the generated image, storing the generated image, modifying a depth of the generated image, or modifying a frequency of the generated image.

17. A method comprising:
receiving, by a processing circuit, location data regarding the location of an external instrument with respect to a tissue imaged by a transducer;
receiving, by the processing circuit, image data of the tissue from the transducer;
generating and displaying, by the processing circuit, based on the image data and the location data, an image on a display device of an ultrasound imaging system, the image comprising the tissue and a representation of the external instrument;
receiving, by the processing circuit, a user input via a user input element disposed on an ultrasound imaging probe of the ultrasound imaging system, the probe comprising the transducer; and
controlling, by the processing circuit, at least one of a display characteristic of the displayed image or an operating characteristic of the transducer based on the user input.

18. The method of claim 17, wherein controlling the at least one of the display characteristic of the displayed image or the operating characteristic of the transducer comprises at least one of: marking a point of intersection between the representation of the external instrument and the tissue, freezing the displayed image, storing the displayed image, modifying a depth of the displayed image, or modifying a frequency of the displayed image.

19. The method of claim 17, wherein the image comprises a point of intersection between the representation of the external instrument and the tissue, wherein the method further comprises:
marking the point of intersection by storing coordinates of the point of intersection, the coordinates relative to a stationary position of a spatial tracking system.

20. The method of claim 17, wherein controlling at least one of the display characteristic of the displayed image or the operating characteristic of the transducer causes a menu to be displayed on the display device, the menu comprising a plurality of options, the plurality of options comprising at least one of: marking a point of intersection between the representation of the external instrument and the tissue, freezing the generated image, storing the generated image, modifying a depth of the generated image, or modifying a frequency of the generated image.
